(19) United States
(12) Patent Application Publication
(10) Pub. No.: US 2012/0285450 A1
(43) Pub. Date: Nov. 15, 2012

(54) DEVICE AND METHOD FOR USE WITH AN ANESTHESIA MACHINE FOR AUTOMATICALLY IDENTIFYING PATIENT INFORMATION

(76) Inventors: Xueqing WANG, Wuxi (CN); Feng Guo, Wuxi (CN)

(21) Appl. No.: 13/473,094

(22) Filed: May 16, 2012

(30) Foreign Application Priority Data
May 6, 2011 (CN) 201110125797.4

(51) Int. Cl. A61M 16/01

(52) U.S. Cl. 128/203.14

(57) ABSTRACT

A device for use with an anesthesia machine for automatically identifying patient information, the device comprising an identification card configured to store patient information and to be associated with a patient, a transmitting module configured to transmit the patient information to the identification card, and an identifying module in communicative connection with a control unit of the anesthesia machine, the identifying module being configured to identify the identification card and to provide the patient information stored in the identification card to the anesthesia machine.

### BACKGROUND OF THE INVENTION

1. Field of the Invention

Embodiments of the present invention relate to a device and a method for use with an anesthesia machine for automatically identifying patient information, and an anesthesia machine comprising the device.

2. Description of the Prior Art

Conventional anesthesia machines cannot automatically obtain patient information that are useful for a more effective or better control of the anesthetic process. In addition, for anesthesia conducted by these anesthesia machines, the control accuracy is not satisfactory.

There exists a need for an anesthesia machine capable of automatically providing patient information data, so as to solve the problem that anesthesia machines may cause harm to target patients under a machine-control mode due to unawareness of physiological characteristics of the patients.

### BRIEF SUMMARY OF THE INVENTION

According to an embodiment of the present invention, there is provided a device for use with an anesthesia machine for automatically identifying patient information. The device comprises an identification card configured to store patient information and to be associated with a patient, a transmitting module configured to transmit the patient information to the identification card, and an identifying module in communicative connection with a control unit of the anesthesia machine, the identifying module being configured to identify the identification card and to provide the patient information stored in the identification card to the anesthesia machine.

According to another embodiment of the present invention, there is provided an anesthesia machine comprising a device for automatically identifying patient information. The device comprises an identification card configured to store patient information and to be associated with a patient, a transmitting module configured to transmit the patient information to the identification card, and an identifying module in communicative connection with a control unit of the anesthesia machine, the identifying module being configured to identify the identification card and to provide the patient information stored in the identification card to the anesthesia machine.

According to another embodiment of the present invention, there is provided a method for use with an anesthesia machine for automatically identifying patient information. The method comprises transmitting patient information to an identification card configured to be associated with a patient, identifying the identification card, and providing the patient information stored in the identification card to the anesthesia machine.

### BRIEF DESCRIPTION OF THE DRAWINGS

Embodiments and aspects of the present invention are described in detail herein and are considered a part of the claimed invention. Other embodiments and aspects can be understood with reference to the following detailed description, accompanying drawings, and claims. In the drawings, identical or substantially identical elements are denoted by same reference numerals, wherein:

FIG. 1 is a structural diagram of a device for use with an anesthesia machine for automatically identifying patient information according to an embodiment of the present invention;

FIG. 2 shows schematically an identifying module capable of identifying an RFID tag according to an embodiment of the present invention;

FIG. 3 illustrates an identifying module in connection with an anesthesia machine according to an embodiment of the present invention;

FIG. 4 provides a flow chart for a method for use with an anesthesia machine for automatically identifying patient information according to an embodiment of the present invention;

FIG. 5 shows schematically a flow chart for actual application of a method for use with an anesthesia machine for automatically identifying patient information according to an embodiment of the present invention.

### DETAILED DESCRIPTION OF THE INVENTION

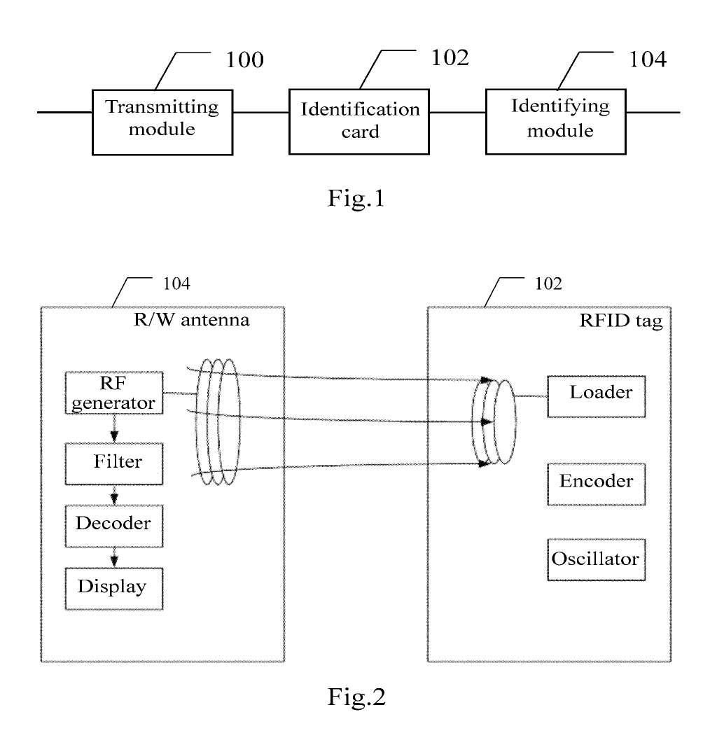

FIG. 1 is a structural diagram of a device for use with an anesthesia machine for automatically identifying patient information according to an embodiment of the present invention, wherein the device primarily comprises a transmitting module 100, an identification card 102, and an identifying module 104.

The transmitting module 100 is for transmitting patient information to the identification card 102. In one example embodiment, the transmitting module 100 comprises, but is not limited to, a hospital information terminal connected with a transmitter, or any other transmitting means capable of sending patient information to the identification card. Said patient information includes name, height, weight, age, sex, basic diseases, physiological characteristics, and the like.

The identification card 102 is for storing the patient information transmitted by the transmitting module 100, and is configured to be adaptive for association with a patient, for example, the identification card is worn by the patient. As long as it is capable of storing patient information and being adaptive to the transmitting module 100 and the identifying module 104 so as to guarantee consistency of communication protocols and information encoding thereof, any information carrier may be suitable for the device for use with an anesthesia machine for automatically identifying patient information according to an embodiment of the present invention. For example, the identification card 102 can be a four-dimensional code or other carriers capable of storing information. In one embodiment, the identification card 102 is a radio frequency identification (RFID) tag.

The identifying module 104 which is in communication with a control unit of the anesthesia machine is used for identifying the identification card 102 and is capable of providing information stored in the identification card 102 to the anesthesia machine. FIG. 2 shows an example of the identifying module 104 capable of identifying the RFID tag 102 according to an embodiment of the present invention, wherein the identifying module comprises a read/write (R/W) antenna, a RF generator, a filter, a decoder, and a display.

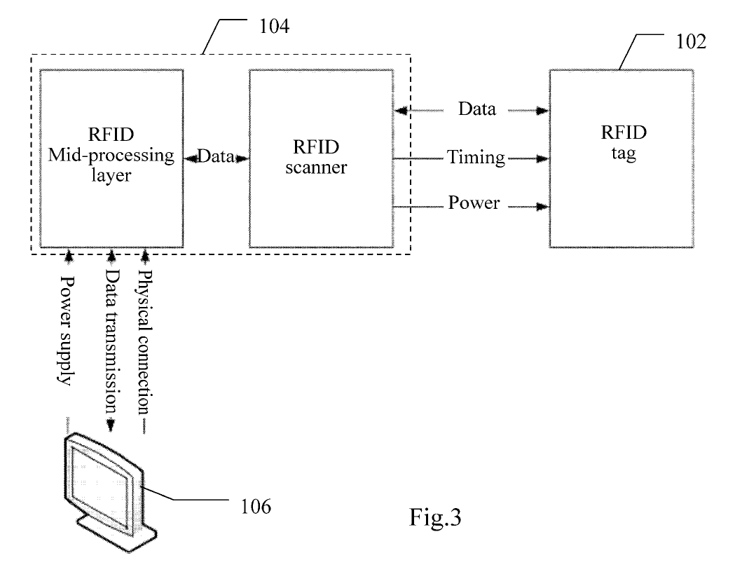

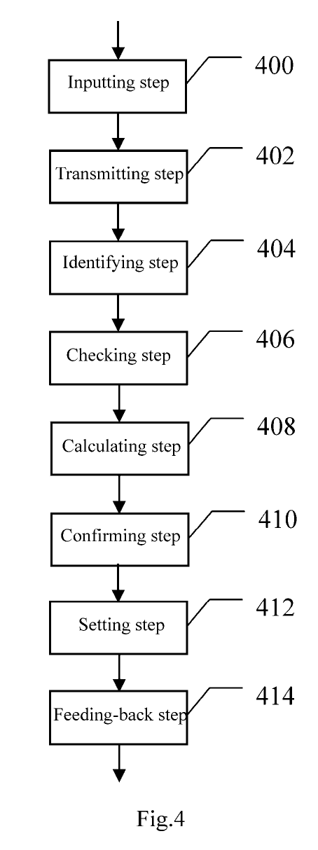

FIG. 3 is a diagram showing the connection between the identifying module 104 and an anesthesia machine 106. In one embodiment, the identifying module 104 is attached to the anesthesia machine 106 (the identifying module 104 is integrated into the anesthesia machine 106).

In another embodiment, the device for use with an anesthesia machine for automatically identifying patient information may optionally comprises a feeding-back module, wherein the anesthesia machine calculates control parameters to be set in accordance with patient information provided, while the feeding-back module feeds the control parameters calculated by the anesthesia machine to a hospital information system terminal, for purposes of statistic collecting, analyzing and processing. This feedback data may be accumulated to optimize correspondingly the current algorithm for auto-setting an anesthetic concentration and a breathing parameter. The feeding-back module may be any transmitting means connected with the anesthesia machine and capable of communicating with the hospital information system terminal. Alternatively, feeding-back is carried out by storing the calculated data, the RFID tag of the patient, or other storage media in time and then forwarding the same to the hospital information system terminal.

The device for use with an anesthesia machine for automatically identifying patient information according to embodiments may be applied on various anesthesia machines, in the form of hardware, software, firmware, or a combination thereof. These applications are readily achievable to one of ordinary skill in the art, and elaboration thereon would be unnecessary herein.

FIG. 4 provides a flow chart for a method for use with an anesthesia machine for automatically identifying patient information according to an embodiment of the present invention, wherein the method primarily includes: a transmitting step 402 and an identifying step 404. In other embodiments, the method may optionally include an inputting step 400, a checking step 406, a calculating step 408, a confirming step 410, a setting step 412, and a feeding-back step 414.

More particularly, the method includes inputting 400 with a hospital information system terminal patient information, for example, the patient's name, height, weight, age, sex, basic diseases, physiological characteristics, and the like, so as to be transmitted to the identification card. The method also includes transmitting 402 the patient information to an identification card configured to be adaptive for association with a patient, identifying 404 the identification card and providing the patient information stored in the identification card to the anesthesia machine. The method includes checking 406 whether the patient information provided is valid, for example, checking data to determine whether it is a valid RFID tag, and then to determine whether the patient information is normal, such as whether the age exceeds 200, and so on. The method also includes calculating 408 control parameters for the anesthesia machine in accordance with the patient information provided, such as an anesthetic concentration and a breathing parameter. Control parameters for the anesthesia machine are displayed for confirming the control parameters, for example, by the doctor. The method further includes setting 412 an alarm value according to control parameters calculated in accordance with the patient information provided, such that the anesthesia machine can trigger a predetermined alarm, for example, in the form of sound, light, etc, when a corresponding parameter exceeds the set alarm value. The method also includes feeding-back 414, by the anesthesia machine, the calculated control parameters to the hospital information system terminal.

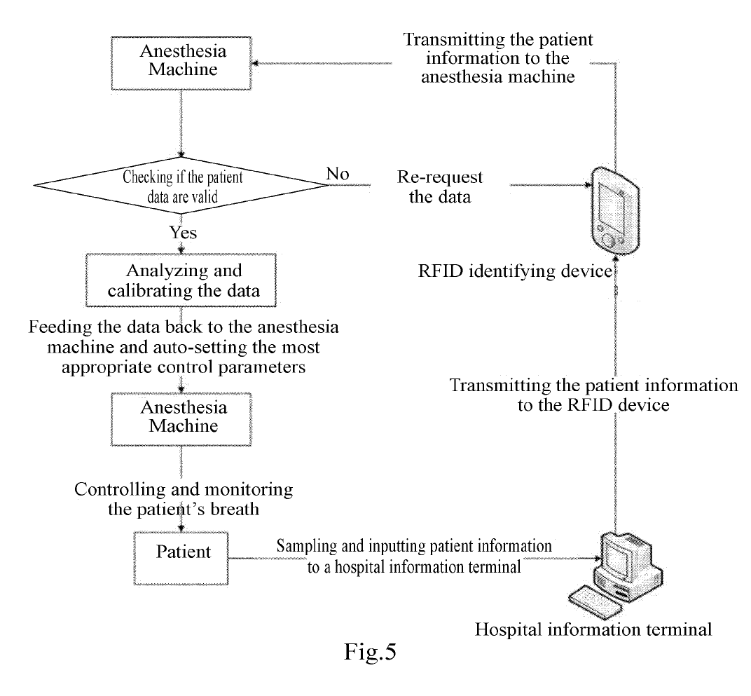

FIG. 5 shows schematically a flow chart for actual application of a method for use with an anesthesia machine for automatically identifying patient information according to an embodiment of the present invention. Before deciding to perform a general anesthetic operation, preoperative evaluation should be conducted. The anesthetist examines the patient's general conditions, basic diseases, current medication, and general anatomical, physiological and mental conditions, and thereby formulates an anesthetic scheme accordingly. During this stage, the anesthetist can input 400 general conditions of the patient (name, height, weight, age, sex, etc), basic diseases (primarily including diseases in connection with the lungs, the respiratory system and the blood system, because these diseases affect respiration and circulation of the patient) and certain physiological characteristics (e.g., such special circumstances as pulmonary fibrosis) in a medical information system terminal of the hospital. Thereafter, the inputted information can be transmitted 402 to and stored in a RFID tag for use in the real operation.

On the day for the operation, the RFID tag is taken out and worn on a hand of the patient in advance. Of course, the RFID tag is not necessarily worn by the patient, as long as the tag will not be lost and is easy for confirmation and identification. When the patient is pushed into the operation room, once this tag is correctly identified 404 by the identifying means of the anesthesia machine, the anesthesia machine can read the data inputted by the anesthetist during inputting 400 from the tag. After the anesthesia machine reads the data, it checks 406 whether the data is valid; for example, it checks data to determine whether it is a valid RFID tag, and then to determine whether the patient information is normal, such as whether the age exceeds 200, and so on. If the checking result is “No”, it re-requests the data; if the checking result is “Yes”, the control unit of the anesthesia machine automatically calculates 408 such data as an anesthetic concentration and a breathing parameter suitable for the patient according to a predetermined algorithm, automatically sets 412 these parameters and displays the same to the anesthetist for confirmation 410. In the meanwhile, an alarm value is set accordingly 412.

Once the anesthetist makes confirmation 410 and the operation starts, the anesthesia machine automatically records states of the patient during the whole operative process and the anesthetist's calibration as to control parameters of the anesthesia machine. Upon successful completion of the whole operation, the anesthetist may choose to store the data in the RFID tag of the patient or store the data in other media, and then to forward them to the medical information system terminal 414 to facilitate analyses, statistics and process. This feedback data may be accumulated to further optimize the current algorithm for auto-setting an anesthetic concentration and a breathing parameter.

By using the device and the method for use with an anesthesia machine for automatically identifying patient information according to embodiments of the present invention, manually inputting control parameters for the anesthesia machine by the doctor are avoided and possible harms to patients under a machine-control mode due to unawareness of physiological characteristics of the patients are prevented, thereby improving control accuracy of the device and reducing clinical risks.

While specific example embodiments of the present invention have been described above, it will be understood by those skilled in the art that various modifications, substitutions and/or changes may be made to the present invention. For example, one step or module in the foregoing example embodiments may be carried out by two or more steps or modules; or, to the contrary, two or more steps or functionalities of two or more modules may be carried out in one step or module. As long as these alterations do not depart from the spirit of the present invention, they shall fall within the scope of the present invention. It is also to be understood that certain terminologies used in the description and claims are illustrative instead of limitative. Moreover, “one example embodiment” and “another example embodiment” in the description refer to different example embodiments, which may, of course, be incorporated, in full or in part, in one example embodiment.

What is claimed is:

1. A device for use with an anesthesia machine for automatically identifying patient information, the device comprising:
an identification card configured to store patient information and to be associated with a patient;
a transmitting module configured to transmit the patient information to the identification card; and
an identifying module in communicative connection with a control unit of the anesthesia machine, the identifying module being configured to identify the identification card and to provide the patient information stored in the identification card to the anesthesia machine.

2. The device for use with an anesthesia machine for automatically identifying patient information according to claim 1, further comprising:
a feeding-back module configured to feed control parameters calculated by the anesthesia machine back to a hospital information system terminal.

3. The device for use with an anesthesia machine for automatically identifying patient information according to claim 1, wherein the identifying module is attached to the anesthesia machine.

4. The device for use with an anesthesia machine for automatically identifying patient information according to claim 1, wherein the identification card is a radio frequency identification tag.

5. The device for use with an anesthesia machine for automatically identifying patient information according to claim 1, wherein the transmitting module comprises a hospital information terminal and a transmitter.

6. The device for use with an anesthesia machine for automatically identifying patient information according to claim 1, wherein the identification card is configured to attach to the patient.

7. The device for use with an anesthesia machine for automatically identifying patient information according to claim 1, wherein the patient information includes name, height, weight, age, sex, basic diseases, and physiological characteristics.

8. The device for use with an anesthesia machine for automatically identifying patient information according to claim 1, wherein the anesthesia machine is configured to calculate and to set control parameters in accordance with the patient information provided.

9. An anesthesia machine comprising a device for automatically identifying patient information, the device comprising:
an identification card configured to store patient information and to be associated with a patient;
a transmitting module configured to transmit the patient information to the identification card; and
an identifying module in communicative connection with a control unit of the anesthesia machine, the identifying module being configured to identify the identification card and to provide the patient information stored in the identification card to the anesthesia machine.

10. A method for use with an anesthesia machine for automatically identifying patient information, the method comprising:
transmitting patient information to an identification card configured to be associated with a patient;
identifying the identification card; and
providing the patient information stored in the identification card to the anesthesia machine.

11. The method for use with an anesthesia machine for automatically identifying patient information according to claim 10, further comprising:
checking whether the patient information provided is valid.

12. The method for use with an anesthesia machine for automatically identifying patient information according to claim 10, further comprising:
calculating control parameters for the anesthesia machine in accordance with the patient information provided.

13. The method for use with an anesthesia machine for automatically identifying patient information according to claim 12, further comprising:
setting an alarm value in accordance with the calculated control parameters.

14. The method for use with an anesthesia machine for automatically identifying patient information according to claim 12, further comprising:
manually confirming the calculated control parameters by displaying the calculated control parameters for the anesthesia machine.

15. The method for use with an anesthesia machine for automatically identifying patient information according to claim 12, further comprising:
feeding the calculated control parameters back to a hospital information system terminal.

16. The method for use with an anesthesia machine for automatically identifying patient information according to claim 10, further comprising:
inputting with a hospital information system terminal the patient information to be transmitted to the identification card.

17. The method for use with an anesthesia machine for automatically identifying patient information according to claim 10, wherein the patient information includes name, height, weight, age, sex, basic diseases, and physiological characteristics.

18. The method for use with an anesthesia machine for automatically identifying patient information according to claim 12, wherein the control parameters include an anesthetic concentration and a breathing parameter.

19. The method for use with an anesthesia machine for automatically identifying patient information according to claim 10, wherein the identification card is a radio frequency identification tag.
